(19) United States
(12) Patent Application Publication
(10) Pub. No.: US 2015/0359848 A1
(43) Pub. Date: Dec. 17, 2015

(54) Methods and Related Kits for Treating and Regenerating Tooth Pulp and Periapical Tissues in a Mammal

(71) Applicant: University of Southern California, Los Angeles, CA (US)

(72) Inventors: Maha Mohamed Fouad Mounir, Alexandria (EG); Malcolm L. Snead, Los Angeles, CA (US)

(21) Appl. No.: 14/715,798

(22) Filed: May 19, 2015

Related U.S. Application Data
(63) Continuation of application No. 12/760,878, filed on Apr. 15, 2010, now abandoned.

(51) Int. Cl. A61K 38/17

(52) U.S. Cl. CPC A61K 38/1709

(57) ABSTRACT

In one embodiment, a method of regenerating tooth pulp in a mammal is provided. The method comprises removing at least a portion the existing tooth pulp of a mammal and adding amelogenin protein to the existing tooth pulp of the mammal to regenerate tooth pulp in a mammal. In another embodiment, a method of treating necrotic tooth pulp of a mammal is provided. The method comprises providing at least one necrotic tooth pulp of a mammal with an amelogenin protein. In yet another embodiment, a kit for regenerating tooth pulp in a mammal is provided. The kit comprises an adequate amount of amelogenin protein for regenerating tooth pulp in a mammal and instructions for applying the amelogenin protein to at least one tooth pulp of the mammal.

### CROSS REFERENCE TO RELATED APPLICATIONS

This application is a continuation application of U.S. patent application Ser. No. 12/760,878, filed on Apr. 15, 2010, which is incorporated herein by reference in its entirety.

### BACKGROUND

1. Field of Disclosure

The present disclosure related generally to a treating and regenerating tooth pulp in a mammal. More specifically, the present disclosure is related to regenerating tooth pulp, pulp tissue containing newly generated blood vessels and pulp cells, periapical bone and portions of the periodontal ligament and treating necrotic tooth pulp in a mammal.

### SUMMARY

In one embodiment of the present disclosure, a method of regenerating tooth pulp in a mammal is provided. The method comprises removing at least a portion the existing tooth pulp of a mammal and adding amelogenin protein to the existing tooth pulp of the mammal to regenerate tooth pulp in a mammal.

In another embodiment of the present disclosure, a method of regenerating tooth pulp in a mammal is provided. The method comprises complete removal of the tooth pulp and adding amelogenin protein to the existing tooth pulp of the mammal or to the periapical area.

In yet another embodiment of the present disclosure, a method of regenerating pulp tissue containing newly generated blood vessels and pulp cells in a mammal is provided. The method comprises adding amelogenin protein to the existing tooth pulp of the mammal to regenerate pulp tissue containing newly generated blood vessels and pulp cells in a mammal.

In yet another embodiment of the present disclosure, a method of regenerating periapical bone and portions of the periodontal ligament in a mammal is provided. The method comprises adding amelogenin protein to the existing tooth pulp of the mammal to regenerate periapical bone and portions of the periodontal ligament in a mammal.

In yet another embodiment of the present disclosure, a method of treating necrotic tooth pulp of a mammal is provided. The method comprises providing at least one necrotic tooth pulp of a mammal with an amelogenin protein.

In yet another embodiment of the present disclosure, a kit for regenerating tooth pulp in a mammal is provided. The kit comprises an adequate amount of amelogenin protein for regenerating tooth pulp in a mammal and instructions for applying the amelogenin protein to at least one tooth pulp of the mammal.

In yet another embodiment, the present disclosure relates to treatment of necrotic tooth pulp and regeneration of healthy tooth pulp of a mammal with recombinant mouse amelogenin protein.

In yet another embodiment, the present disclosure relates to regeneration of healthy tooth pulp and periapical regeneration in dogs.

In an aspect of at least one embodiment of the present disclosure, the packet platelets have been removed from the added amelogenin protein.

In another aspect of at least one embodiment of the present disclosure, the added amelogenin protein is recombinant amelogenin protein.

In yet another aspect of at least one embodiment of the present disclosure, the added amelogenin protein is rM180.

In yet another aspect of at least one embodiment of the present disclosure, the amelogenin protein is an isoform LRAP.

In yet another aspect of at least one embodiment of the present disclosure, the portion of the existing tooth pulp of the mammal that is removed is inflamed, infected or necrotic.

In yet another aspect of at least one embodiment of the present disclosure, the amelogenin protein is a protein from 60 to 180 amino acids in length.

In yet another aspect of at least one embodiment of the present disclosure, an applicator is provided to apply the amelogenin to the tooth pulp or other area needing treatment and/or regeneration.

In yet another aspect of at least one embodiment of the present disclosure, the amelogenin is recombinant amelogenin (r-amelogenin) and the r-amelogenin treats periapical infection in a mammal.

### DRAWINGS

The above-mentioned features and objects of the present disclosure will become more apparent with reference to the following description taken in conjunction with the accompanying drawings wherein like reference numerals denote like elements and in which:

FIG. 1 shows the hematoxylin and eosin micrograph of the 6-month period obtained from the experiments described herein and visually demonstrates some of the benefits and advantages of the claimed inventions.

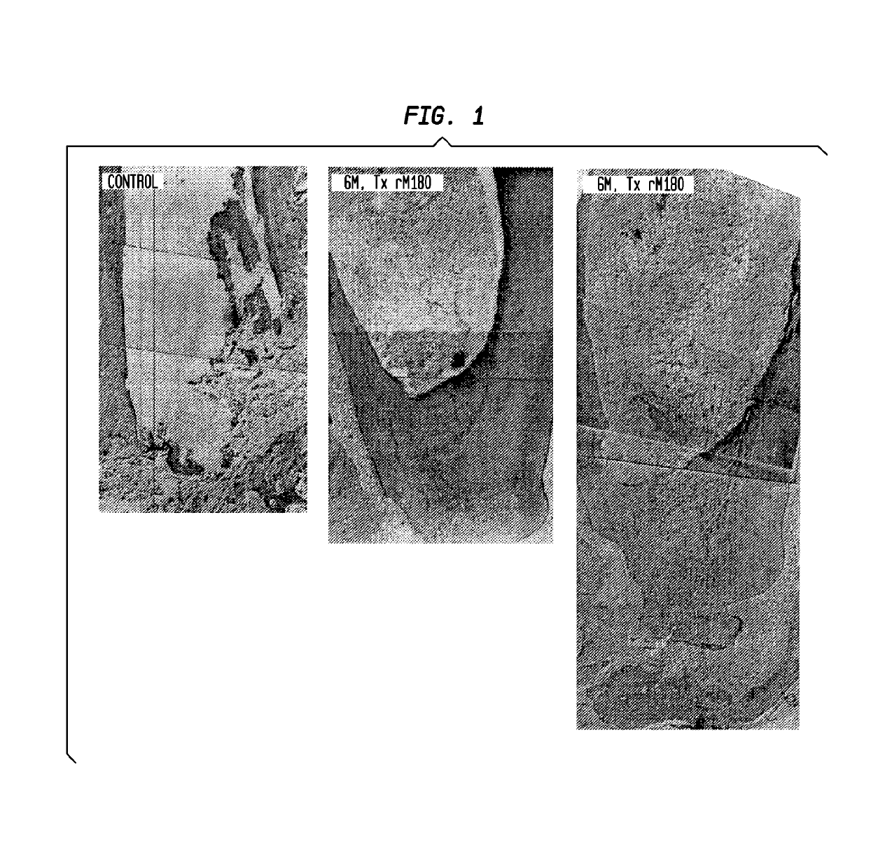

FIG. 2 shows a micrograph of the apical area of a tooth treated with recombinant r-amelogenin protein after 6 months. Among other things, the results show a complete regeneration of the attachment apparatus including bone, dentin, periodontal ligament and cementum. Additionally, the results show regeneration of the pulp tissue with blood vessels and pulp cells. The trichrome stained micrograph of the tissue 6-montha after the treatment period obtained from the experiments described herein and visually demonstrates some of the benefits and advantages of the claimed inventions.

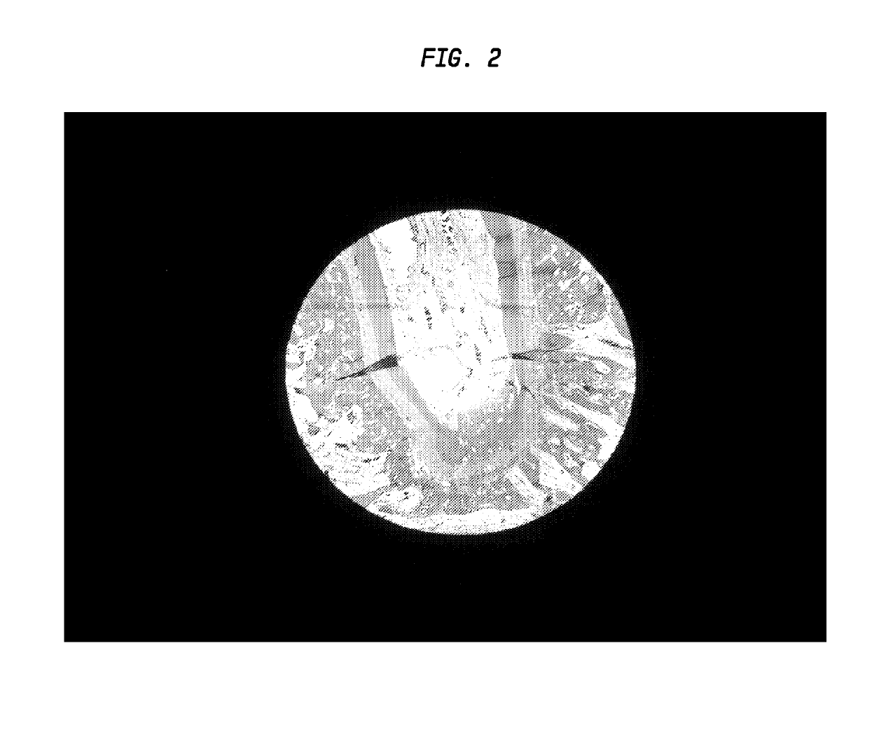

### DETAILED DISCLOSURE

Experiments were initiated to determine if recombinant mouse 180 amino acid long amelogenin protein (rM180) would regenerate tooth pulp and it was this therapy that was a useful adjunct for apexification (closure of the root apex) in experimental animals.

In one set of experiments, a total of 8 mongrel breed dogs were used. Ten upper and ten lower molars on each dog were treated endodontically, resulting in a total of 160 root canals (2 canals for each molar). Initially, all root canals were left open to the oral flora without coronal restorations for 14 days and all became contaminated, resulting in the death of the pulp tissue in all of the 160 teeth. All 160 root canals were then cleaned, irrigated and filled with either: 1) rM180 amelogenin (80 canals) or 2) calcium hydroxide, the traditional filling material (80 root canals).

After 1-month, a nearly completely calcified tissue barrier was formed in the canals treated with rM180 amelogenin. In addition, regeneration of pulp tissue containing newly regenerated blood vessels, newly regenerated pulp cells and dental tissues was demonstrated. Moreover, regeneration of the periapical bone and portions of the periodontal ligament that were lost as a result of inflammation were demonstrated. In contrast, nearly no calcified barrier formed in the calcium hydroxide treated group with only remnants of necrotic pulp debris was observed. By 3 to 6 months, the group treated with rM180 showed regeneration of viable, mature pulp tissue with closure of the apical foramen and regeneration of the bone and the periodontal ligament. The canals treated with calcium hydroxide showed incomplete apical formation characterized by occasional islands of dentine at the periapical area.

It should be appreciated that as part of the experiment all of the pulp tissue was removed at the time of the initial endodontic treatment. Furthermore, the pulp chambers were allowed to become contaminated by oral microorganisms for 14-days, a condition that is predicted to result in the death of any and all pulp remnants. In addition, these conditions result in inflammation to the periapical area from necrotic tissue and microorganisms caused the resorption of periapical bone and destruction of the periapical periodontal ligament.

The mechanical removal of the pulp and the microbial contamination resulted in the death of the pulp with inflammatory changes to adjacent periapical tissues resulting in their destruction.

The regeneration of the cells contributing to closure of the apex and restoration of the adjacent bone and periodontal ligament did not originate from residual remnants of the pulp.

It is believed that adult stem cells were recruited by the rM180 amelogenin and these stem cells contributed to the regeneration of pulp, bone and the periodontal ligament providing the regenerated tissues, including the restoration of a complete pulp tissue.

The health of a human tooth depends on the integrity of the dental hard tissues and the support of living soft tissues. The enamel is the outer hard layer of the tooth which is supported by another layer of hard tissue, the dentin. Dentin surrounds the pulp of the tooth, the living soft tissue that contains cells, blood vessels, nerves, and fibrous matrix. The pulp-dentin border is lined by odontoblasts, which are derived from pulp. These highly specialized cells are responsible for the production and repair of dentin. Even if the pulp is encased in hard tissue it can be exposed by caries or by trauma.

Caries exposures are common and cause inflammation or death of the tooth pulp. These conditions are treated by procedures usually referred to as endodontic (or root canal) treatment. The root canal treatment itself and even more the following restorative procedures will remove a substantial amount of tooth structure. A significant advancement in endodontic treatment would be to support or restore the regenerative and repair properties of dental pulp and treat any affected or necrotic areas. The present disclosure provides these and other related benefits and advantages.

It should be appreciated that the restoration of the tooth can then be done with minimal loss of structure and the methods and kits described here will revolutionize current treatment of exposed dental pulps. More specifically, instead of extracting the inflamed pulp tissue, the methods and related kits of the present disclosure can be used. Using the methods and kits of the present disclosure, the regenerated pulp will re-build a damaged tooth from the inside. A tooth with a regenerated pulp will require markedly smaller to no restorations and a tooth with a regenerated pulp may uphold the proprioceptive function of the tooth and withstand masticatory forces without additional coronal restorations.

A survey of dental practices by the American Dental Association estimates that approximately 24 million root canal therapies are performed each year in the United States. With a very conservative average cost of $400 per treatment this represents $9.6 billion per year for endodontic therapy alone. The tooth structure then must be restored with available dental materials. The restorative costs are approximately 14.4 billion dollars for an average individual restoration cost of $600. The methods and related kits of the present disclosure provide methods to avoid/reduce these costs.

In one embodiment of the present disclosure, a method of regenerating tooth pulp in a mammal is provided. The method comprises removing at least a portion the existing tooth pulp of a mammal and adding amelogenin protein to the existing tooth pulp of the mammal to regenerate tooth pulp in a mammal.

In another embodiment of the present disclosure, a method of regenerating pulp tissue containing newly generated blood vessels and pulp cells in a mammal is provided. The method comprises adding amelogenin protein to the existing tooth pulp of the mammal to regenerate pulp tissue containing newly generated blood vessels and pulp cells in a mammal.

In yet another embodiment of the present disclosure, a method of regenerating periapical bone and portions of the periodontal ligament in a mammal is provided. The method comprises adding amelogenin protein to the existing tooth pulp of the mammal to regenerate periapical bone and at least portions of the periodontal ligament in a mammal.

In yet another embodiment of the present disclosure, a method of treating a necrotic tooth pulp of a mammal is provided. The method comprises providing at least one necrotic tooth pulp of a mammal with an effective amount of amelogenin protein.

In yet another embodiment of the present disclosure, a kit for regenerating tooth pulp in a mammal is provided. The kit comprises an adequate amount of amelogenin protein for regenerating tooth pulp in a mammal and instructions for applying the amelogenin protein to at least one tooth pulp of the mammal.

In yet another embodiment, the present disclosure relates to treatment of necrotic tooth pulp and regeneration of healthy tooth pulp of a mammal with recombinant mouse amelogenin protein.

In yet another embodiment, the present disclosure relates to regeneration of healthy tooth pulp and periapical regeneration in dogs.

In an aspect of at least one embodiment of the present disclosure, the packet platelets have been removed from the added amelogenin protein.

In another aspect of at least one embodiment of the present disclosure, the added amelogenin protein is recombinant amelogenin protein.

In yet another aspect of at least one embodiment of the present disclosure, the added amelogenin protein is rM180.

In yet another aspect of at least one embodiment of the present disclosure, the amelogenin protein is an isoform LRAP.

In yet another aspect of at least one embodiment of the present disclosure, the portion of the existing tooth pulp of the mammal that is removed is inflamed, infected or necrotic.

In yet another aspect of at least one embodiment of the present disclosure, the kit also includes an applicator, which is well known in the art, to apply the amelogenin protein to the tooth and/or tooth pulp.

In yet another aspect of at least one embodiment of the present disclosure, the amelogenin protein is a protein from 60 to 180 amino acids in length.

In yet another aspect of at least one embodiment of the present disclosure, at least one applicator is provided to apply the amelogenin to the tooth pulp or other area needing treatment and/or regeneration.

While the methods and kits disclosed herein have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

What is claimed is:

1. A method of regenerating tooth pulp in a mammal, the method comprising:
(a) removing at least a portion the existing tooth pulp of a mammal; and
(b) adding amelogenin protein to the existing tooth pulp of the mammal to regenerate tooth pulp in a mammal.

2. The method of claim 1, wherein the packet platelets have been removed from the added amelogenin protein.

3. The method of claim 1, wherein the added amelogenin protein is recombinant amelogenin protein.

4. The method of claim 1, wherein the added amelogenin protein is rM180.

5. The method of claim 1, wherein the amelogenin protein is an isoform LRAP.

6. The method of claim 1, wherein the portion of the existing tooth pulp of the mammal that is removed is inflamed, infected or necrotic.

7. The method of claim 1, wherein the amelogenin protein is a protein from 60 to 180 amino acids in length.

8. A method of treating necrotic tooth pulp of a mammal, the method comprising providing at least one necrotic tooth pulp of a mammal with an amelogenin protein.

9. The method of claim 8, wherein the packet platelets have been removed from the amelogenin protein.

10. The method of claim 8, wherein the amelogenin protein is recombinant amelogenin protein.

11. The method of claim 8, wherein the amelogenin protein is rM180.

12. The method of claim 8, wherein the amelogenin protein is an isoform LRAP.

13. The method of claim 8, wherein the portion of the existing tooth pulp of the mammal that is removed is inflamed, infected or necrotic.

14. The method of claim 8, wherein the amelogenin protein is a protein from 60 to 180 amino acids in length.

15. A kit for regenerating tooth pulp in a mammal, the kit comprising:
(a) an adequate amount of amelogenin protein for regenerating tooth pulp in a mammal; and
(b) instructions for applying the amelogenin protein to at least one tooth pulp of the mammal.

16. The kit of claim 15, wherein the packet platelets have been removed from the amelogenin protein.

17. The kit of claim 15, wherein the amelogenin protein is recombinant amelogenin protein.

18. The kit of claim 15, wherein the amelogenin protein is rM180.

19. The kit of claim 15, wherein the amelogenin protein is an isoform LRAP.

20. The kit of claim 15, wherein the amelogenin protein is a protein from 60 to 180 amino acids in length.
